(19) United States
(12) Patent Application Publication
(10) Pub. No.: US 2007/0243102 A1
(43) Pub. Date: Oct. 18, 2007

(54) Method and Plant for the Treatment of Fibrous Material Susceptible to Degradation by Biological Activity

(76) Inventor: Peter Rainer Philipp, Charlotte, NC (US)

Correspondence Address: OSTROLENK FABER GERB & SOFFEN, 1180 AVENUE OF THE AMERICAS, NEW YORK, NY, 100368403, US

(21) Appl. No.: 11/574,693

(22) PCT Filed: Sep. 6, 2004
(86) PCT No.: PCT/CH04/00558

§ 371 Date: Mar. 5, 2007

(51) Int. Cl. A61L 2/20; B32B 5/02

(52) U.S. Cl. 422/27; 422/296; 422/33; 442/152

(57) ABSTRACT

Material susceptible to degradation by biological activity, before all by microorganisms like bacteria and fungi, is treated with ozone. The ozone penetrates the material and not only deactivate the active organisms themselves, but also spores of bacteria and fungi. The ozonization is performed in at least one cycle consisting of subjecting the material to underpressure, e.g. of 500 mBar, in a vessel, then filling the vessel with ozone containing gas. In a preferred embodiment, the ozonization may be preceded by at least one steaming cycle, consisting essentially of applying vacuum, then filling the vessel with steam, notably with steam of about 80° C.

The present invention relates to a method for treating fibrous material prone to degradation by biological activity, particularly cotton bales, and to a plant for executing the method.

For trade and transport purposes, raw cotton is pressed into bales. Presently, the cotton bales are defined to have 8.5% water content inter alia because their price is fixed by weight. However, being a biological material, and supported by this water content, the cotton is prone to rotting, i.e. biological degradation by bacteria, fungi etc.

U.S. Pat. No. 6,557,267 (Wanger; filed on 27 Apr. 2001) which is hereby incorporated by reference, describes a method for suppressing or at least retarding the rotting process of cotton bales. The bales are repeatedly subjected alternatingly to vacuum and “water gas”, i.e. essentially steam (“Steaming”). Thereby, the steam penetrates deeply into the bales driven by the pressure difference between bale core and the outside of the bale. Usually, the steam has a temperature of about 80° C. (“steaming temperature”) and is applied with a pressure equal to the vapour pressure of water at the steaming temperature which is about 0.5 Bar.

The process is conducted in a manner that even the core of the bales reaches a temperature of about 80° C. At this temperature, most active microbes are destroyed or deactivated. However, e.g. spores of fungi survive, and accordingly, growth of fungi will occur again some time later, e.g. after 1 to 2 months.

Similar rotting or degradation effects are observed with yarns and textile materials consisting at least partly of fibrous material susceptible to such degradation. In particular, yarns are traded with a water content of 8.5%, too, which promotes biological activity.

It is an object of the present invention to propose a method providing an ameliorated protection of fibrous material, particularly of cotton bales, prone to degradation by biological activity, more particularly by the activity of microbes including fungi.

Accordingly, the microbes including their spores are more efficiently and substantially destroyed or inactivated by ozone penetrating the fibrous material, or more precisely by a gas having an effective concentration of ozone. Particularly, the material may be bales of (raw) cotton.

Within the whole specification including the claims, by the word “microbes”, any kind of microscopic organisms are meant, like bacteria and fungi, either in its active form or spores. By spores, the durable forms of bacteria and fungi are meant.

Percentages are given by weight if not otherwise indicated.

Preferably, the treatment by ozone, the so-called ozonisation, is performed after a steam treatment, e.g. according to U.S. Pat. No. 6,557,267.

According to claim 14, a plant for executing the method comprises a vessel for receiving the fibrous material, a venting or vacuum pump, and an ozone generator. Generally, for practical reasons, ozone destroying means and air purging means are needed in order to establish acceptable working conditions by purging the atmosphere in the vessel. The latter means are needed for complying with requirements of working conditions and environmental protection.

As mentioned above, in a preferred embodiment, a plant according to the invention additionally comprises the equipment needed for the steam treatment.

The invention will now be further described by means of an non-limiting exemplary embodiment with reference to the drawing.

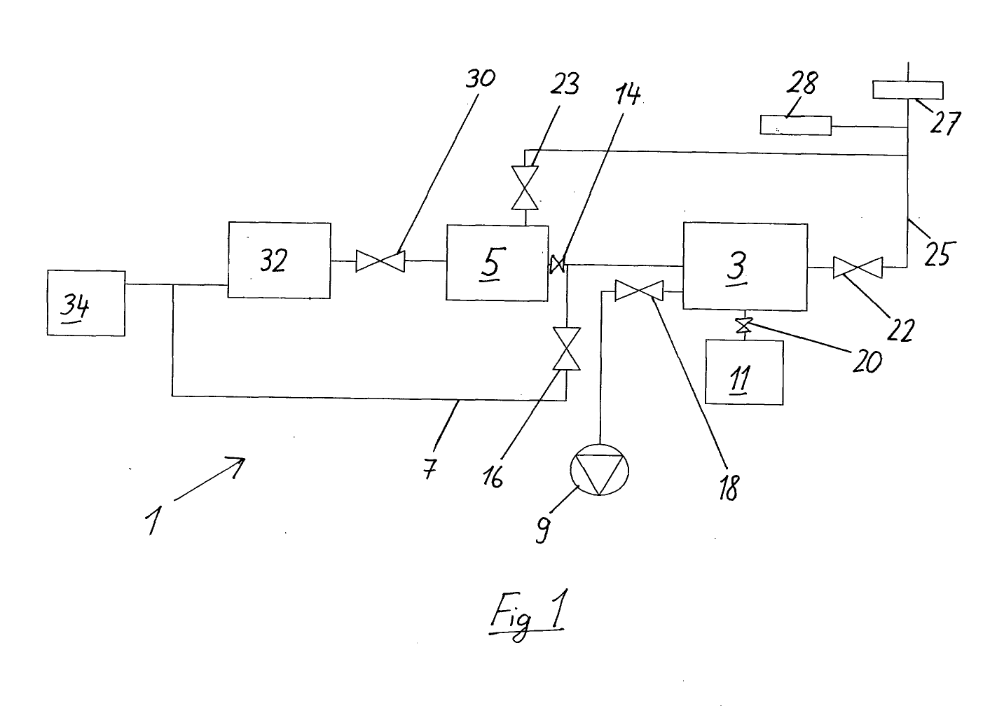

FIG. 1 shows a scheme of a plant according to the invention;

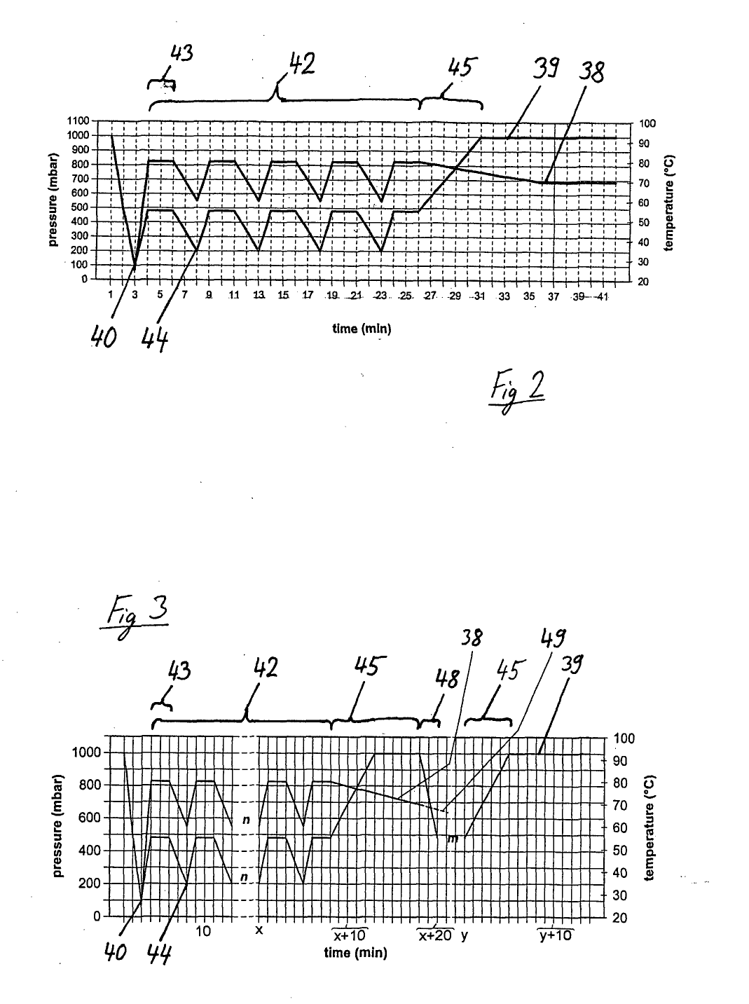

FIG. 2 shows curves of temperature and pressure vs. time for a first execution manner; and

FIG. 3 curves of temperature and pressure vs. time for a second execution manner.

Structure

The plant 1 for performing the method is arranged around a vessel 3 which is filled by the material to be treated. The material may be bales of raw cotton, but also uncolored cotton yarn bobbins, cotton textile pieces, webs, felts, fleeces or the like. Instead of pure cotton fibrous materials, other natural fibrous materials like ramie, sisal, jute, flax, wool, silk, either as pure material or in combination with cotton may be considered, too, which are susceptible to biological degradation. As a rule, these materials are of biological origin.

The vessel 3 is connected to an ozone reservoir 5, a purging gas conduit 7, a vacuum pump 9, and a steam generator 11 via respectively an ozone supply valve 14, a purging valve 16, a vacuum valve 18, and a steam valve 20.

The vessel 3 and the ozone reservoir 5 are connected via respective exhaust valves 22, 23 to the exhaust line 25. The exhaust line 25 is provided with a vent ozone destructor (VOD) unit 27 and a sensor 28 for determining the ozone concentration in the exhaust line 25. The vent ozone destructor unit 27 reduces the ozone concentration to a degree that the exhaust gas may be discharged into the environment.

The exhaust valve 23 of the ozone reservoir 5 allows to dispose ozone in case of overpressure or to exchange its contents entirely after a working interruption or for long non-using periods.

The ozone reservoir 5 is supplied with ozone via an ozone generator valve 30 by the ozone generator 32. The ozone generator 32 is of a known type, e.g. one using a dielectric barrier discharge. The ozone generator 32 is supplied with the purging gas as the working gas. The gas is furnished by a gas supply 34.

As an alternative, the ozone generator 32 may have its own gas source, e.g. pure oxygen for producing a gas stream of elevated ozone concentration to the reservoir 5. The purging gas may be air which is purified in order to avoid a secondary contamination with microorganisms of the treated material during the purging step. If the gas source 34 serves the ozone generator 32, the gas needs to comply additionally with the requirements of the latter, i.e. it has to have a dew point of e.g. −60° C. at most and must be free of dust and hydrocarbons. For instance, with pure oxygen, an ozone concentration of up to about 14% can be reached, whereas with air, at most about 4.5% of ozone are feasible with ozone generators of the dielectric barrier discharge type.

In order to keep the ozone reservoir 5 small, it is filled with ozone containing gas under elevated pressure. Hence, the ozone generator is of a type furnishing ozone containing gas of at least 1 Bar overpressure.

Operation

The material to be treated, e.g. cotton bales, is stored in the vessel 3 and the vessel 3 is closed.

FIG. 2 shows the development of temperature 38 [° C.] and pressure 39 [mBar] versus time [minutes] during the treatment. In an initial phase, a first vacuum 40 of 100 mBar is produced in the vessel 3 by opening the vacuum valve 18. Thereby, the initially contained air is removed as much as possible. Of course, higher organisms, like insects, may already be killed thereby.

In the second phase, one to five cycles of steaming during time 42 are performed. First, the steaming valve 20 is opened for flooding the vessel 3 with steam. After about 2 minutes, the steam valve 20 is closed and the vacuum valve 18 is opened to reduce the pressure again to about 200 mBar within about 2 minutes. During the steaming periods 43, the pressure in the vessel is about 500 mBar, and the temperature is about 80° C. As mentioned above, the pressure is determined by the vapor pressure of water at the selected steaming temperature, e.g. 80° C. Still to be noted that the temperature in the core of the bales of cotton, particularly if compacted as is usually the case, reaches about 80° C. in the last steaming period 43 only.

In other terms, the steaming cycles are repeated as often, and the ratio of steaming duration and withdrawal of steam by vacuum is chosen the way that at least during the last period, the so-called core temperature of the units of treated material (e.g. cotton bales) reaches the steaming temperature.

Still to be observed that the temperature in the vessel decreases to about 60° C. at the end 44 of each underpressure period. This temperature is, however, arbitrary and merely occasioned by the thermal characteristics of the system (insulation, time needed for establishing the reduced pressure, evaporation etc.).

The fifth steaming period is followed by the ozonization phase 45 which takes about 5 minutes and during which the pressure in the vessel attains ambient pressure (1 Bar). The vessel is filled with ozone containing gas from the ozone reservoir 5 by opening the ozone supply valve 14. As ozone generators in reasonable size are not capable of furnishing the needed volumes of ozone in only a few minutes with reasonable efforts, the time between the ozonization phases is used for filling the ozone reservoir 5 using an ozone generator of lower output rate.

As the pressure in the vessel 3 is still about 500 mBar or less, the ozone is drawn into the material. Furthermore, as long as there is free ozone available it remains active in the inner parts of the material and continues to deactivate microorganisms and spores etc., particularly also during the following phases.

After the ozonization phase 45, the vessel 3 is purged by opening the purge valve 16. The purging process, normally with purified air or oxygen furnished by the gas source 34, is maintained until the ozone sensor 28 indicates that the vessel may be opened without danger.

The purge gas valve 16 is closed and the vessel 3 is opened. The material is removed and wrapped in an about microorganism-tight packaging, e.g. a foil. The packaging retains the ozone containing atmosphere in the material. Formerly, the material have already been packed in a sealed package in order to maintain the water content of 8.5%, hence often the conventional packaging step may be sufficient, possibly slightly modified to ameliorate microorganism-tightness.

As the ozone has a sterilizing and anti-microbial effect as well within the material, the long-term danger is merely re-contamination from the environment. However, besides the packaging, the periphery of the material itself has a filtering effect and impedes penetration and secondary contamination by microorganisms and spores. Particularly, the feared rotting in the core of the material (e.g. cotton bales) which is almost invisible from the outside is effectively suppressed.

FIG. 3 shows a generalized execution manner of the process according to the invention in a representation like FIG. 2 with the same numerals designating corresponding steps. Here, n steaming cycles are executed, followed by m ozonization cycles, with m+n=total number of cycles. Specifically, the m ozonization steps are each preceded by an underpressure period 48 during which the pressure within the vessel 2 is lowered to a pressure between 100 and about 500 mBar. As the ozone containing gas is relatively cool, the temperature decreases during the ozonisation cycles and approaches ambient temperature, indicated by the cut broken line 49.

After the m ozonization cycles, the procedure as described above with reference to FIG. 2 subsequent to ozonization may follow analogously.

From the above said, further variants are conceivable to the one skilled in the art, like alternatingly steaming and ozonising several times. However, the last step is an ozonization step so that the advantage of the effect of ozone remaining within the wrapped material, e.g. cotton bales is maintained.

Still to mention that the ozone may also have a bleaching effect which may be an additional advantage at least in the case of treating cotton bales.

Another aspect is that ozone even kills higher organisms like insects. Particularly in border-crossing trading, a special and often environmentally critical treatment like fumigation with e.g. methylbromide, cyanic acid or other biocides may be avoided.

From the examples set forth above, the one skilled in the art is able to derive numerous variants and alterations without leaving the scope of protection of the invention which is defined by the claims, for example:
The invention may be applied to compacted and uncompacted raw cotton, other fibrous materials of biological origin prone to degradation, rotting etc. by microorganisms and other living organisms, or materials containing these fibrous materials including cotton in admixture with other components, e.g. synthetic fibers or wool.
Application to yarn and thread bobbins or textile objects, particularly of significant thickness or arranged in dense stacks. Like cotton bales, yarn and thread bobbins are often required to have a water content of 8.5% which promotes growth of microorganisms.
Application of ozonization without prior steaming by repeatedly applying vacuum and flooding with ozone. After purging, the treated material is again put into a microorganism-tight packaging or wrapping to keep the ozone inside of the material.
Particularly for strongly compacted materials, more than one ozonization cycle may be applied like described with reference to FIG. 3, yet for better penetration, the underpressure may be emphasized in reducing the pressure precedingly to ozonization to a pressure lower than 500 mBar, preferably in the range of 100 mBar to 500 mBar.
When the vessel is filled with ozone, a predetermined pause at final pressure may follow to allow the ozone concentration within the material to better equilibrate, and only then the purging is performed. Inversely, with respect to the generalized method, one or more of the ozonization steps may be followed immediately by an underpressure step. E.g. the plateau period of the first ozonization step 45 (left in FIG. 3) is shortened or suppressed, and the last of the ozonistion steps 45 (right in FIG. 3), is extended by a wait time before purging.
Generally, the duration of the ozonization is to be adapted to the actual requirement, e. g. to the kind, density and thickness of the material to be treated. It has been found that the ozonization duration may be chosen between about 3 minutes to about 20 minutes, from the start of filling the vessel until the start of the next step (creation of underpressure, purging etc.).

1. Method for treating fibrous material prone to degradation by biological activity, wherein the material is subjected at least once to an ozonization cycle comprising the steps of
subjecting the material to underpressure in a closed vessel, and subsequently
applying an ozonization step to the material, the ozonization step comprising filling the vessel with a gas having an effective ozone concentration, optionally followed by leaving the material in the ozone containing atmosphere, so that the material is effectively penetrated by the gas in order to destroy microorganisms in the material by the ozone.

2. Method according to claim 1, wherein the underpressure is a pressure of at most about 500 mBar (50 kPa).

3. Method according to claim 1, wherein the underpressure is a pressure of about 100 mBar (10 kPa) to about 500 mBar (50 kPa).

4. Method according to claim 1, wherein the ozonisation step has a duration of from 3 minutes to 20 minutes.

5. Method according to claim 1, wherein at least the final ozonization step has a duration of about 5 minutes.

6. Method according to claim 1, wherein the ozone content of the ozone containing gas is at least 4 wt.-%.

7. Method according to claim 1, wherein the ozone containing gas is produced by passing a gas consisting of air up to pure oxygen through an ozone generator converting oxygen into ozone.

8. Method according to claim 7, wherein the ozone containing gas is stocked in an ozone reservoir and the vessel is filled substantially by emptying the ozone reservoir into the vessel.

9. Method according to claim 1, wherein the material comprises compacted or uncompacted raw cotton bales.

10. Method according to claim 1, wherein the material is bales, bobbins and/or pieces of textile webs, fabrics, threads, felts, fleeces and/or yarns consisting at least partially of cotton and/or other fibrous materials susceptible to degradation by biological activity.

11. Method according to claim 1, wherein after completion of the ozonization, the material is wrapped in an essentially microorganism-tight packaging in order to keep the ozone within the material.

12. Method according to claim 1, wherein preceding or interspersed with the ozonization, the material is subjected to a steaming treatment comprising at least one cycle with the steps of
subjecting the material to underpressure in a closed vessel
filling the vessel with steam in order to heat the material thoroughly to a temperature where biological organisms are destroyed or inactivated.

13. Method according to claim 12, wherein the last steaming cycle is followed by at least one final ozonization cycle, the creation of underpressure of the first of the final ozonisation steps being optionally constituted partially or totally by the underpressure prevailing at the end of the preceding steaming cycle.

14. Plant for performing the method according to claim 1, comprising a vessel capable to be essentially gas-tight closed, the vessel being connected to at least an ozone source and a vacuum pump.

15. Plant according to claim 14, comprising additionally a steam source, so that the vessel can be filled with steam.

16. A fibrous material prone to degradation by biological activity, wherein the material is wrapped in essentially microorganism-tight packaging and the free space within the material is filled with a gas containing ozone in order to suppress the biological activity.

17. The fibrous material according to claim 16, wherein the material is a wrapped cotton bale, bobbins and/or pieces of textile webs, fabrics, threads, felts, fleeces and/or yarns consisting at least partially of cotton and/or other fibrous materials susceptible to degradation by biological activity.
